(19) United States
(12) Patent Application Publication
(10) Pub. No.: US 2016/0095499 A1
(43) Pub. Date: Apr. 7, 2016

(54) Colon Capsule with Textured Structural Coating for Improved Colon Motility

(71) Applicant: Capso Vision, Inc., Saratoga, CA (US)

(72) Inventors: Mikael Trollsas, San Jose, CA (US); Mark Hadley, Newark, CA (US); Ganyu Lu, Palo Alto, CA (US); Kang-Huai Wang, Saratoga, CA (US)

(73) Assignee: Capso Vision, Inc.

(21) Appl. No.: 14/504,458

(22) Filed: Oct. 2, 2014

(51) Int. Cl. A61B 1/00; A61B 1/06; A61B 5/00; H04N 5/225; A61B 1/04

(52) U.S. Cl. CPC A61B 1/00075; H04N 5/2251; H04N 5/2252; A61B 1/041; A61B 5/42; A61B 1/06; A61B 1/0002; A61B 1/00016; A61B 1/0011; H04N 2005/2255

(57) ABSTRACT

The present invention discloses a capsule device with textured structural surface so that the capsule device has desired surface properties when it travels through designated regions in the gastrointestinal tract. The capsule device according to the present invention comprises a sensor and a capsule housing, where the sensor is sealed in the capsule housing. The capsule housing having a textured surface to cover at least one region of an exterior surface of the capsule housing to increase holding force between the luminal wall and the capsule device when the capsule device travels inside a gastrointestinal (GI) tract after being swallowed. A coating can be added to cover the textured surface so as to make the capsule surface smooth for easy to swallow. The coating layer will dissolve when the capsule device is in contact with the acid fluid inside the GI tract.

### CROSS REFERENCE TO RELATED APPLICATIONS

The present invention is related to U.S. Pat. No. 7,983,458, entitled “in vivo Autonomous Camera with On-Board Data Storage or Digital Wireless Transmission in Regulatory Approved Band”, granted on Jul. 19, 2011. The U.S. Patent is hereby incorporated by reference in its entirety.

### FIELD OF THE INVENTION

The present invention relates to diagnostic imaging inside the human body or any other living creature. In particular, the present invention relates to an in-vivo capsule that has textured surfaces for improved motility through the gastrointestinal (GI) tract.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician or a veterinary physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot easily reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage. The capsule device 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. Capsule device 110 includes battery power supply 24 and an output port 26. Capsule device 110 may be propelled through the GI tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls (100) on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine the images and correctly identify any anomaly.

When the capsule device travels through the GI tract, the capsule device will encounter different environments. It is desirable to manage the capsule device to travel at a speed that sufficient sensor data (e.g., images) can be collected at all locations along the portions of the GI tract which are of interest, without wasting battery power and/or data storage by collecting excessive data in some locations. In order to manage the capsule device to travel at a relatively steady speed, techniques have been developed to change the capsule specific gravity during the course of travelling through the GI tract. In some environments, it is desirable to have a capsule with higher specific gravity. In other environments, it may be desirable to have a capsule with lower specific gravity. For example, it is desirable to configure the capsule device to have a lower specific gravity when the capsule device travels through the ascending colon. On the other hand, it may be desirable to configure the capsule device to have a higher specific gravity when the capsule device travels through the descending colon if the descending colon is filled with liquid. However, techniques based on specific gravity or density control may not work reliably due to various reasons. For example, the change of specific gravity or density may not have to take place at the intended section of the GI tract. Therefore, the location of the capsule device inside the GI tract has to be monitored or estimated. However, the location of the capsule device usually cannot be accurately determined without the use of additional equipment outside the patient's body. Therefore, it is desirable to develop reliable means to manage the capsule device to travel at a relatively steady speed in the GI tract.

### BRIEF SUMMARY OF THE INVENTION

The present invention discloses a capsule device with a textured structural surface so that the capsule device has desired surface properties when it travels through designated regions in the gastrointestinal tract. The capsule device according to the present invention comprises a sensor and a capsule housing, where the sensor is sealed in the capsule housing. The capsule housing having a textured surface to cover at least one region of an exterior surface of the capsule housing to increase holding force between the luminal wall and the capsule device when the capsule device travels inside the gastrointestinal (GI) tract after being swallowed.

In one embodiment, the sensor corresponds to an image sensor of a camera to capture images in the GI tract. The capsule device further comprises a light source to illuminate the luminal wall, and the camera further comprises lenses to project the images onto the image sensor. The light source and the lenses are also sealed in the capsule housing. The capsule device can be of an elongated shape having a longitudinal axis and two ends in the direction of the longitudinal axis. The camera can be configured to have one or more lenses located in a middle section of the capsule device in the longitudinal axis. In this case, the textured surface covers at least a portion of the two ends and leaves the corresponding middle section of the capsule housing un-covered to avoid obstructing the field of view of the camera. The camera may also be configured to have a lens located at one or both ends of the capsule device in the longitudinal axis. In this case, the textured surface covers at least a portion of the middle section of the capsule device in the longitudinal axis and leaves the corresponding end of the capsule housing un-covered to avoid obstructing the field of view of the camera. The capsule device may also include an on-board storage to store the captured image or a wireless transmitter to transmit the captured images to an external wireless receiver. The capsule device may also be configured to have lenses at one or both of the longitudinal ends in combination with having one or more lenses in the middle section of the device.

One aspect of the present invention addresses the texture patterns for the textured capsule device. If the capsule device has an elongated shape with a longitudinal axis, the textured surface may use one or more texture patterns selected from a group consisting of a set of dots or circles, a set of loop lines around the longitudinal axis, a set of multi-directional line segments, a set of curved lines in a slant plane with respect to the longitudinal axis, a set of curved lines in a plane parallel with the longitudinal axis, and a set of wavy loop lines around the longitudinal axis. The textured surface can be created from one or more texture patterns raised or recessed from a nominal surface of the capsule housing. The texture patterns raised or recessed from the nominal surface of the capsule housing can be formed by removing or adding a material to the nominal surface of the capsule housing.

Most capsules are adapted to have a smooth exterior surface for easy to swallow. The textured capsule device will help the device to move up in the ascending colon faster, moving through the transverse colon, and/or to slow down in the descending colon. Therefore, the capsule device travels in the GI tract with a steadier pace. Nevertheless, the textured surface may make it harder to swallow. To overcome this issue, another embodiment of the present invention adds a coating to cover the textured surface so as to make the capsule surface smooth again. The coating layer will cover at least a portion of the texture surface and the coating layer can be made of a material that dissolves, degrades or becomes separated from the capsule housing when the capsule device is in contact with the acid fluid inside the GI tract. The coating layer can be made from an enteric material that dissolves in high pH level environment including terminal ileum, cecum, small intestines and colons in the GI tract. For example, the enteric material can be selected from a group consisting of polymers, polysaccharides, plasticizers, methyl cellulose, gelatin and/or sugar. The coating layer may also be made from a low-pH dissolvable material that dissolves in low pH level environment including stomach. For example, the low-pH dissolvable material can be selected from a group consisting of ethylene glycol, polyethylene glycol, vinyl alcohol, polyvinyl alcohol, vinylpyrrolidone, polyvinylpyrrolidone, carboxy methyl cellulose, hyaluronic acid, sodium chloride (NaCl), potassium chloride (KCl), sodium carbonate (Na2CO3), potassium carbonate (K2CO3) and sodium bicarbonate (NaHCO3).

### BRIEF DESCRIPTION OF THE DRAWINGS

FIG. 1 shows schematically a capsule camera system in the GI tract, where archival memory is used to store captured images to be analyzed and/or examined.

FIG. 2A-FIG. 2B illustrate examples of a capsule device incorporating surface texture control achieved in-vivo by the use of an enteric or dissolvable top-coating, where the capsule device with the textured surface is shown in FIG. 2A and the textured surface covered by the coating is shown in FIG. 2B.

FIG. 3A-FIG. 3E illustrate examples of texture patterns, where a set of isotropic dots or circles is shown in FIG. 3A, a set of multi-directional line segments is shown in FIG. 3B, with a set of curved lines in planes parallel to the longitudinal direction of the capsule is shown in FIG. 3C, a set of curved in slant planes (e.g. diagonal) and/or a gradient pattern with respect to the longitudinal axis is shown in FIG. 3D and a set of wavy loop lines around the longitudinal axis is shown in FIG. 3E.

FIG. 4A-4B illustrate examples of a capsule device incorporating surface structure by removing materials from existing surface of the capsule housing, where an isotropic surface structure (i.e., dots or circles) is shown in FIG. 4A and the surface structure with grove loop lines around the longitudinal axis is shown in FIG. 4B.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

For a capsule device with an image sensor, it is critical to have a steady and consistent travelling velocity inside different regions of the GI tract (e.g. stomach, small bowel, ascending and descending colons) so that sufficient and stable images and video can be obtained. The travelling velocity of the capsule camera depends on many factors including regional gastrointestinal motility, gravitational force, buoyancy and viscous drag of the surrounding fluids. After the capsule deice is swallowed, it is propelled into the esophagus. Peristaltic waves in the esophagus and gravitational force move the camera into the stomach. After the capsule device passes the cardia and enters the stomach with fluid, the balance among gravitational force, buoyancy and drag from the gastric fluids starts to affect its travelling velocity and transit time. After the capsule enters the stomach, the movement of the capsule device is also affected by the migrating myoelectric cycle (MMC), which is a cyclically occurring pattern of electric and mechanical activity. The MMC can be divided into four phases. Phase 1 lasts between 30 and 60 minutes with rare contractions. Phase 2 lasts between 20 and 40 minutes with intermittent contraction. Phase 3, or housekeeping phase, lasts between 10 and 20 minutes with intense and regular contractions for short period. The housekeeping wave sweeps all the undigested material out of the stomach to the small bowel. Phase 4 lasts between 0 and 5 minutes and occurs between phase 3 and phase 1 of two consecutive cycles. For the capsule device to travel aborally at a desired velocity in all four phases, preferably phases 1 and 2, its specific gravity needs to be greater than 1 (e.g., 1.1) to overcome the buoyance and drag from the surrounding fluid. If phase 3 is detected through image motion detection or accelerometer, the specific gravity can be pushed to a value less than one (e.g., 0.95) for the capsule device to float to the top and to retake the video in a more stable phase.

In the small intestine, BER (basic electrical rhythm) is around 12 cycles per minute in the proximal jejunum and decreases to 8 cycles per minutes in the distal ileum. There are three types of smooth muscle contractions: peristaltic waves, segmentation contractions and tonic contractions. Normally, peristalsis will propel the capsule device towards large intestines. Since the small intestine twists and turns around between the stomach and the large intestine, the capsule device may sometimes be trapped at corners and turns. In this case, motion detection may be used to detect such situation. Accordingly, density, or specific gravity-changing mechanisms can be used to slightly change the balance between gravity and buoyancy so that the capsule device can leave the trap sooner before the next peristalsis. For example, a capsule device using multi-phase density control is disclosed in PCT Patent Application, Serial No. PCT/US13/66011, filed on Oct. 22, 2013

While the large intestine is one organ, it demonstrates regional differences. The proximal (ascending) colon serves as a reservoir and the distal (transverse and descending) colon mainly performs as a conduit. The character of the luminal contents impacts the transit time. Liquid passes through the ascending colon quickly, but remains within the transverse colon for a long period of time. In contrast, a solid meal is retained by the cecum and ascending colon for longer periods than a liquid diet. In the ascending colon, retrograde movements are normal and occur frequently. Capsule endoscopy is typically performed with ambulatory patients whose torsos are erect for a majority of the time. The transit of a dense capsule is hastened in certain environment of the GI tract, such as the descending colon where the capsule device moves down in the direction of gravity, if the capsule coating is polished and slippery. On the other hand, when the capsule device move up in the ascending colon against the direction of gravity, the capsule device may get stuck near the lower end of the ascending colon (i.e., near the cecum) if the capsule device is dense and the capsule coating is polished and slippery. The travel against the direction of gravity may also happen in the transverse colon as the typical human transverse colon anatomy, as well described by virtual colonoscopy, has significant curvature in the coronal plan moving back and forth in the directions of the superior and inferior parts.

In order to reliably manage the capsule device to travel at a steady speed in the GI tract, it is needed for the capsule density control based approach to change the density or the specific gravity so that the buoyant force may overcome the gravitational force and retropulsion. However, the capsule density based approach may not be accurate. Therefore, embodiments of the present invention adopt a total different approach from the capsule density control. Instead, the present invention causes textured surface on the capsule device to allow the luminal side of the intestinal wall to hold on to the capsule device as it is moving up the ascending or the transverse colon in the direction against gravity and/or as it is moving down the descending colon or the transverse in the direction of gravity. The textured surface on the capsule device according to the present invention will cause the capsule device to move up the ascending colon faster and/or to move down the descending colon slower. Consequently, a steadier travel speed in the GI tract is accomplished. The textured capsule device can be made from a regular capsule device having a smooth surface. A regular capsule device having a smooth surface is referred as a nominal capsule device in this disclosure. A regular capsule housing having a smooth surface is referred as a nominal capsule housing in this disclosure. The present invention may also be used jointly with the capsule density or capsule specific gravity control based approach.

In order to configure the capsule device with a proper structure, it is plausible to change the surface of a nominal capsule housing by adding, removing, or moving material around. For a capsule device, the components are usually sealed in a housing, where the shape and the surface of the housing are adapted for easy swallowing. The texture formation process can be applied to the housing and the process can be done mechanically, chemically, or through ablation using external energy such as lasing. The use of chemicals allows application of a coating for etching the surface. This can be combined by using photolithography as a method to modify the surface. Photolithography combines coating and etching by using a negative or positive photoresist. Both positive and negative as well chemically amplified photolithography methods and materials can be used to create texture patterns on the capsule surface. The texture patterns may also be created on the surface by deforming the surface using heated elements.

The textured surface may use various patterns to increase the holding force with the luminal intestinal wall. For example, the surface of the capsule device according to one embodiment of the present invention is textured with a topography scale in the micrometer to millimeter range and some texture may have different height or depth than other parts of the texture.

While the textured surface helps the capsule device to move at a steadier speed inside the GI tract, it is desirable to have a smooth surface for the capsule device for easy swallowing. Therefore, a coating is applied to the textured capsule device to make the surface smooth before the capsule device is swallowed according to another embodiment of the invention. Once the device enters the stomach of the intestines, the coating starts to dissolve, degrade or become separated from the capsule housing. For example, the coating material can be selected so that when the capsule enters the large intestine, the topographic surface structure will be exposed. Therefore, the textured surface will become in contact with the luminal side of the intestinal wall to allow the capsule device to be gripped and be transported in a more controlled rate. In the descending colon and rectum, propulsive contractions prevail. The capsule device is carried aborally towards the rectum by the natural propulsion. Accordingly, the surface texture of the capsule device as disclosed can help to shorten the transit time and allow a smooth and steady motion.

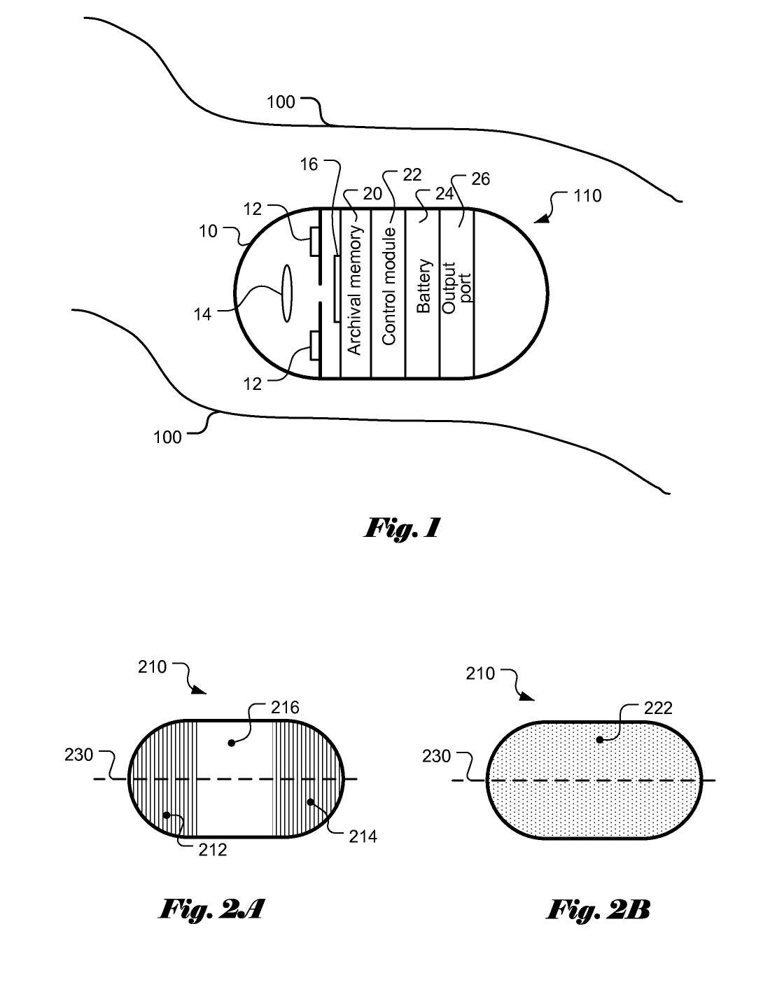

FIG. 2A illustrates an example of a capsule device (210) having textured surface according to an embodiment of the present invention. The textured surface is applied to two regions (212 and 214) toward the two ends in the longitudinal direction (230) of the capsule device. The capsule device in this example includes panoramic cameras located in the middle section of the capsule device along the longitudinal axis. In order to avoid obstructing the fields of view for the cameras, the whole middle section or portions of the middle section is designated as a keep-out out area (216), where the textured surface will not be applied. In order to allow the capsule device with the textured surface to be swallowed easily, the textured surface is coated with materials to form a smooth surface (222) throughout the exterior surface of the capsule device as shown in FIG. 2B before the capsule device is administered. Alternatively, the coating is only applied to the regions with textured surface. The coating will allow the capsule device to transform from a polished easy-to-swallow state (FIG. 2B) before it is swallowed to a rough topography state (FIG. 2A) after it is swallowed so that the capsule device can be easily gripped by the luminal side of the intestinal walls. In one embodiment, the rough topography that can be transformed from a polished state is coated with a low-pH dissolvable coating. In another embodiment, the coating is an enteric coating, which will remain intact in stomach and other areas of the GI tract with low pH values. However, the enteric coating will dissolve when it approaches the terminal ileum or the cecum, where the pH value rises to a higher level.

In one embodiment, the dissolvable coating comprises a drug such as bisacodyl, lubiprostone, and/or linaclotidine or a chemical that is released in the intestines to accelerate colonic transit. The dissolvable or enteric coating may cover the entire capsule device or may only cover the textured surface. These dissolvable coatings are designed to dissolve in the stomach or small bowel within a short period after swallowing such as about 30 to 90 minutes. For the enteric coating, the coating will not dissolve in the low pH of the stomach. However, when the capsule device with enteric coating enters the higher pH environment of the small bowel or colon, the coating will disintegrate. The enteric coatings may be made of polymers, polysaccharides, plasticizers, methyl cellulose, gelatin, sugar, or other materials. Methacrylic acid co-polymer type C is an example of an enteric polymer.

In another embodiment, said surface texture control could be accomplished in-situ by the use of an enteric or dissolvable top-coating. Alternatively, it could be accomplished in-situ through the use of a phase separated polymer matrix material, where the polymer matrix would be mixed with a dissolvable organic or inorganic material. Examples of organics would be hydrophilic polymers such as ethylene glycol, polyethylene glycol, vinyl alcohol, polyvinyl alcohol, vinyl pyrrolidone, polyvinyl pyrrolidone, carboxy methyl cellulose, hyaluronic acid, or similar or readily degradable materials such as PLGA. Examples of inorganics would be various types of salts such as sodium chloride (NaCl), potassium chloride (KCl), sodium carbonate (Na2CO3), potassium carbonate (K2CO3) and sodium bicarbonate (NaHCO3).

The texture formation may cover an entire capsule exterior or only cover partial exterior. For example, the textured surface can be in the longitudinal ends of the capsule device as shown in FIG. 2A when the cameras are located in the middle section of the capsule device. Alternatively, the textured surface can be applied to the middle section of the capsule in the longitudinal direction or the equator of the capsule device when the camera or cameras are mounted in the front and/or rear end of the capsule device. In this case, the textured surface is applied to region 216 in FIG. 2A.

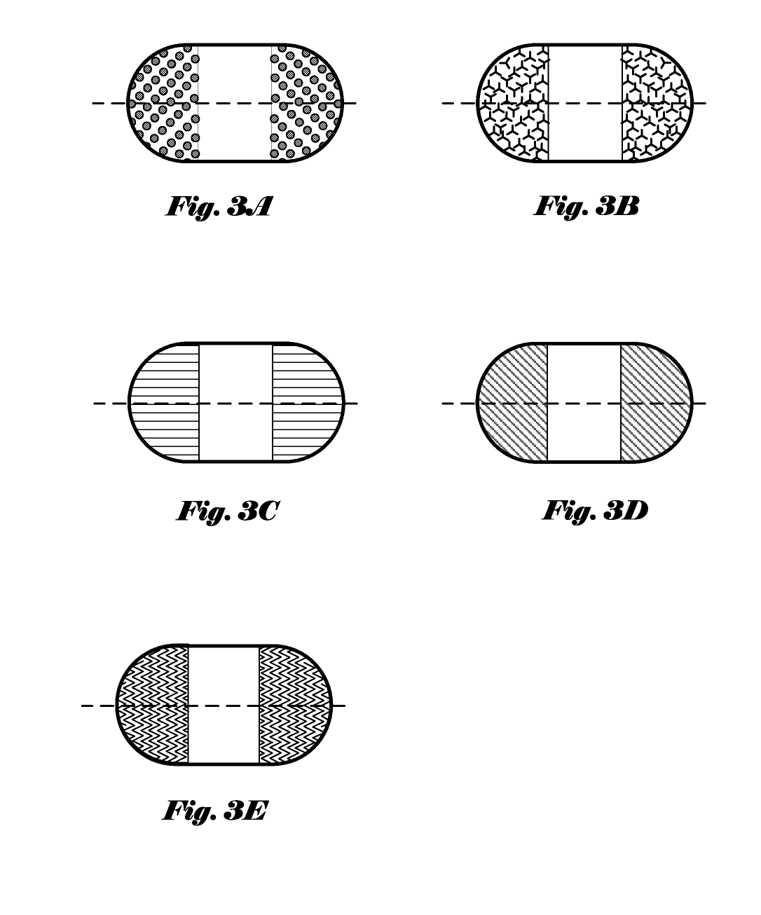

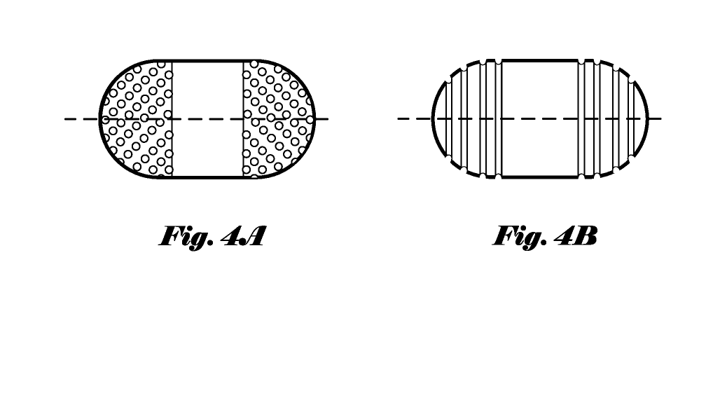

In the example shown in FIG. 2A, the texture pattern corresponds to loop lines around the longitudinal axis having a plane perpendicular to the longitudinal axis of the capsule device. While loop lines are shown in FIG. 2A, one or more helical lines may also be used, where the helical line or lines go around the longitudinal axis. Other texture patterns may also be used to accomplish the goal of causing more gripping force for the capsule device. For example, the texture can be isotropic such as dots or circles as shown in FIG. 3A or have structure in multiple directions such as multi-directional line segmented as shown in FIG. 3B. While three multi-directional line segments are joined at one end, the multi-directional line segments may also be disjoined. Furthermore, different number of line segments may be joined as well.

The structure pattern may also be uni-directional. For example, the texture may correspond to curved lines in planes parallel to the longitudinal direction of the capsule as shown in FIG. 3C. The curved lines may also be in slant planes (e.g. diagonal) and/or a gradient pattern with respect to the longitudinal axis as shown in FIG. 3D. Whether isotropic or structured, the texture may have a topography made from particles, bumps, lines, polygons or random patterns. Furthermore, wavy loop lines as shown in FIG. 3E or a pattern with zig-zag nature may also be used. In FIG. 3E, the triangular wave lines around the longitudinal axis are used as an example of wavy loop lines. Other wavy loop line such as a sine wave may also be used to go around the longitudinal axis.

While various examples of textured patterns are illustrated in FIG. 3A through FIG. 3E, these examples are never meant for an exhaustive list of texture patterns for the present invention. A person skilled in the art may alter the texture pattern to exercise the present invention. For example, instead of using regular dots as shown in FIG. 3A, porous surface-like dots may also be used. Furthermore, the three-connected-segments pattern in FIG. 3B may be replaced by other featured pattern to practice the present invention. While all the texture patterns are applied to two longitudinal ends of the capsule device in FIG. 3A through FIG. 3E, the textured patterns may also be applied to other regions of the capsule device to avoid possible obstruction of the camera field of view or for other concerns.

As mentioned earlier that if the capsule device stays in the ascending colon for too long, the battery may be exhausted before the capsule device finishes its intended tasks, such as capturing images of the colon. Therefore, a capsule device having a textured surface is disclosed such that the luminal side of the intestinal wall of the colon can easily grab the capsule device and cause the capsule device to pass the ascending colon faster. On the other hand, when the capsule device passes to the descending colon, the capsule device having a textured structure designed will slow down in the descending colon so that the capsule device may be able to capture sufficient data (such as images). Accordingly, in another embodiment, the design of the capsule surface texture has directions that allow the ascending or the transverse colon to hold on to the device, as it is moving up the ascending or the transverse colon in the direction against gravity. Furthermore the same capsule surface texture also has a design that allows the capsule device to slow down in the descending or the transverse colon as it travels in the direction of gravity. Therefore, the design of the capsule surface texture is disclosed to allow the capsule to travel through all the different sections of the GI tract at a proper pace.

When applying the pattern or the coating (e.g., enteric or dissolvable coating) to the capsule housing, the coating material may not be able to stay firmly on the capsule housing. When such material is used, to the capsule housing is first coated with a primer to alter the adhesion to the housing surface. The primer coating may be applied across the whole capsule. The primer coating may also be applied to selected areas depending on the optical properties as well as the mechanical and adhesive properties. The selected areas may correspond to the two ends of the capsule in the longitudinal direction while leaving the middle section free from coating so as not to affect the optical transparency of the housing and the ability to capture good images. PMMA, PBMA, polyzene or other hydrophobic polymers or copolymers (block or random) such as PLDA are examples of useful, elastic hydrophobic primers with good adhesion to capsule materials such as polycarbonate.

In another embodiment, the capsule device is coated with a material to cause the capsule slippery, i.e., having a reduced friction (comparing to case without the coating) with the body lumen or the gastric fluid. The reduced friction will allow the capsule device to travel faster under the peristalsis force so to reduce procedure time. Furthermore, slipperiness will reduce the chance that the capsule device gets trapped at corners and turns in the intestines. Hydrophilic coatings are one type of coating that increases lubricity in an aqueous medium.

The present invention can be applied to in-vivo capsule camera applications, either with on-board storage or with wireless transmission system. Both applications will be benefitted from the textured surface since the capsule device will travel through the GI tract at a more reliable pace. The present invention may also be ideal for other capsules indicated for pH- or pressure measurements or any other type of diagnostics or therapy.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1-16. (canceled)

17. A trimodal polyethylene consisting essentially of three polymeric weight fractions A,B,C, wherein the low molecular weight fraction A is a homopolymer and the medium and the high molecular weight fractions B and C, respectively, are copolymers of ethylene and 1-butene as the comonomer,
the polyethylene consists essentially of 50 to 60% (w/w) of homopolymer A, 22 to 26% (w/w) of copolymer B, 18 to 24% (w/w) of copolymer C, and 0 to 5% (w/w) of non-polymeric additives and/or polymeric lubricants selected from the group consisting of: (i) colorants, (ii) antioxidants; (iii) stabilizers; (iv) inorganic or carbonic acids or acid anhydrides; (v) non-polymeric lubricants; (vi) a fluoropolymer lubricant; and (vii) polybutene-1, based on the total weight of the polymer, and wherein the polyethylene is obtained by stepwise polymerization in the presence of a solid Ziegler-Natta catalyst component, where the solid catalyst is the product of a process comprising (a) reacting magnesium diethoxide with titanium tetrachloride carried out in a hydrocarbon at a temperature of 50-100° C., (b) subjecting the reaction mixture obtained in (a) to a heat treatment at a temperature of 110° C. to 200° C. for a time ranging from 3 to 25 hours (c) isolating and washing with a hydrocarbon the solid obtained in (b), said solid catalyst component having a Cl/Ti molar ratio higher than 2.5,
wherein the polyethylene has a density of 0.954 to 0.960 g/cm³, a melt index (HLMI) according to ASTM D-1238, at 190° C. and 21.6 kg, of 2.9 to 4.2 g/10 min and a swell ration of 151 to 182%, and
the polyethylene is produced by polymerization with a Ziegler-Natta catalyst.

18. (canceled)

19. The trimodal polyethylene of claim 17, wherein the stepwise polymerization is carried out in such a way, optionally using a prepolymerized catalyst, that in a first step, the homopolymer A is obtained having a melt index according to ASTM D-1238, at 190° C. and 21.6 kg, of from 18 to 30 g/10 min, and wherein in a second step, copolymer B is obtained the polymer mixture in the reactor having a melt index according to ASTM D-1238, at 190° C. and 21.6 kg, of from 8 to 14 g/10 min, and wherein in a third step, copolymer C is obtained, the polymer mixture of A, B and C in the reactor having a melt index according to ASTM D-1238, at 190° C. and 21.6 kg, of from 3 to 6 g/10 min.

20. The trimodal polyethylene of claim 17, wherein the stepwise polymerization is carried out in three reactor steps wherein at least the first two reactor steps are carried out in suspension and wherein the last reactor step is carried out in a gas phase or suspension reactor.

21. The trimodal polyethylene of claim 17, having a dimensionless ratio of HLMI:MI₅ of from 16 to 23, wherein MI₅ is the melt index according to ASTM D-1238, at 190° C. and 5 kg.

22-24. (canceled)

25. The trimodal polyethylene of claim 17, wherein the reaction of the magnesium alcoholate with TiCl₄ is carried out at a molar ratio of Ti/Mg in the range 1.5 to 4, at a temperature from 60 to 90° C. and for a time of 2 to 6 hours.

26. The trimodal polyethylene of claim 25, wherein the Ti/Mg ranges from 1.75 to 2.75.

27. The trimodal polyethylene of claim 17, wherein the heat treatment in step (b) is carried out at a temperature ranging from 100 to 140° C., for a period of time ranging from 5 to 15 hours.

28. The trimodal polyethylene of claim 17, wherein the Cl/Ti molar ratio is at least 3.

29. The trimodal polyethylene of claim 17, wherein the solid obtained after (c) has the following composition:
Mg:Ti:Cl=1:0.8-1.5:3.2-4.2.

30. The trimodal polyethylene of claim 17, wherein the solid catalyst component is further contacted in a step (d) with an aluminum alkyl halide compound selected from dialkylaluminum monochlorides of the formula R₂³AlCl or the alkylaluminum sesquichlorides of the formula R₃³Al₂Cl₃ in which R³ can be identical or different alkyl radicals having 1 to 16 carbon atoms.

31. The trimodal polyethylene of claim 30, wherein the aluminum alkyl halide is an aluminum alkylchloride compound, and wherein the aluminum alkylchloride compound is used in amounts such that the Al/Ti molar ratio, calculated with reference to the Ti content of the solid catalyst component as obtained by the previous step, is from 0.05 to 1.

32. (canceled)

33. A process comprising blow molding the trimodal polyethylene of claim 17.

34. The trimodal polyethylene of claim 17, wherein the stepwise polymerization further comprises the presence of trialkylaluminum as a cocatalyst component.

35. The trimodal polyethylene of claim 19, wherein in the stepwise polymerization, the partial pressure of 1-butene is controlled at 3 to 10% of that of ethylene in the gas phase of a reactor in the second step.

36. The trimodal polyethylene of claim 19, wherein the melt index of the polymer mixture is from 4 to 5 g/10 min.

37. (canceled)
